(19) United States
(12) Patent Application Publication
(10) Pub. No.: US 2013/0085304 A1
(43) Pub. Date: Apr. 4, 2013

(54) PROCESSES FOR PREPARATION OF POLYMORPHIC FORMS OF LACOSAMIDE

(71) Applicants: Mukesh Kumar Madhra, Karnal (IN); Hari Mohan Sriram, Krishna (IN); Mukesh Kumar Sharma, Gurgaon (IN); Satish Chandra Jha, Muzaffarpur (IN)

(72) Inventors: Mukesh Kumar Madhra, Karnal (IN); Hari Mohan Sriram, Krishna (IN); Mukesh Kumar Sharma, Gurgaon (IN); Satish Chandra Jha, Muzaffarpur (IN)

(73) Assignee: RANBAXY LABORATORIES LIMITED, New Delhi, Delhi (IN)

(21) Appl. No.: 13/510,981

(22) PCT Filed: Nov. 19, 2010
(86) PCT No.: PCT/IB2010/002963

§ 371 Date: Oct. 24, 2012

(30) Foreign Application Priority Data
Nov. 19, 2009 (IN) 2385/DEL/2009

(51) Int. Cl. C07C 231/00

(52) U.S. Cl. CPC C07C 231/00
USPC 564/159

(57) ABSTRACT

The present invention relates to processes for the preparation of crystalline polymorphic forms of lacosamide (Formula I), including processes for inter-conversion among such polymorphic forms.

### FIELD OF THE INVENTION

The present invention relates to processes for the preparation of crystalline polymorphic forms of lacosamide. The processes also provide inter-conversion of these polymorphic forms.

### BACKGROUND OF THE INVENTION

Lacosamide (SPM 927, also referred to as harkoseride or ADD 234037), is chemically (R)-2-acetamido-N-benzyl-3-methoxypropionamide and represented by Formula I. It shows excellent effects to the treatment of pain, epilepsy, fibromyalgia syndrome, osteoarthritis and migraine. It is also known to be useful for the treatment of CNS disorders in humans.

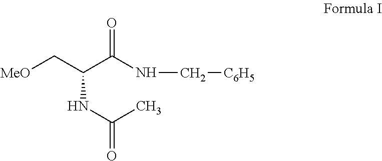

Lacosamide is available in the U.S. market as solution and tablet dosage forms with the proprietary name of Vimpat®. The tablets are indicated as adjunctive therapy in the treatment of partial-onset seizures in patients with epilepsy aged 17 years and older. The solution (injection) dosage form is useful when an oral administration is temporarily not feasible.

Lacosamide and its methods of preparation are disclosed in U.S. Reissue Pat. No. 38,551 (hereinafter referred to as the '551 patent). In this patent, lacosamide is isolated according to conventional techniques; more precisely, according to the examples described, it is obtained either by filtration of crude lacosamide with diethyl ether or purified by flash column chromatography. The '551 patent makes no reference to the existence of polymorphic forms of lacosamide.

An alternative method for the preparation of lacosamide is disclosed in PCT publication WO 2006/037574 (hereinafter referred to as '574 application) that comprises exchanging dichloromethane with ethyl acetate through distillation of a solution of lacosamide in dichloromethane and crystallizing lacosamide by cooling to 0° C. to 5° C. The polymorphic form obtained by following the process exemplified in the '574 application is herein designated as Form-A. FIG. 1 represents the X-ray Powder Diffraction pattern (XRPD) of Form A of lacosamide which is obtained by following the process described in the '574 application.

An article published at IP.com (Reference: IPCOM000187362D) mentions crystallization of lacosamide with 2-propanol at 50° C. to produce the said Form A of lacosamide. A characteristic XRD pattern of the Form A is also provided in the article.

Our published U.S. Patent Application 2009/0143472 describes preparation of lacosamide. Examples 9 and 10 of this application provides crystalline polymorphic Form B and Form A, respectively.

The marketed lacosamide product with the brand name Vimpat® (200 mg) has been found to contain crystalline polymorphic Form A of lacosamide.

### SUMMARY OF THE INVENTION

Aiming to develop a new and efficient process for the preparation of polymorphic form(s) of lacosamide in pure form, the present inventors attempted various crystallization methods with low to high volume of solvents at several temperatures with a certain amount of lacosamide. To their surprise, it was found that some batches of lacosamide after crystallization with ethyl acetate provided crystalline polymorphic Form A, whereas other batches provided crystalline polymorphic Form B of lacosamide. This observation suggested that the volume of solvent(s) taken for crystallization plays an important role in generating the polymorphic form.

The present inventors worked on crystallization techniques and found that dilution, temperature and volume of solvent(s) plays an important role in obtaining crystalline polymorphic forms of lacosamide in pure form.

Thus, the present invention relates to various processes for the preparation of crystalline polymorphic Forms A and B of lacosamide.

### DESCRIPTION OF TERMS

The term “low volume of ethyl acetate”, herein refers to about 7 to about 12 times of ethyl acetate by volume in milliliters per gram of lacosamide.

The term “high volume of ethyl acetate”, herein refers to 12 times or more by volume in milliliters of ethyl acetate per gram of lacosamide.

The term “crude lacosamide”, herein refers to the lacosamide which is not crystallized or purified but isolated from the reaction medium wherein it is chemically synthesized. This can be in a dried or wet condition.

The term “about”, herein when used along values assigned to certain measurements and parameters means a variation of 10% from such values, or in case of a range of values, means a 10% variation from both the lower and upper limits of such ranges. In case of DSC and melting point data the term about means±2° C.

The term “ambient temperature”, herein refers to the temperature of surroundings where the experiment is performed. For example, ambient temperature can vary from 15° C. to 35° C.

The processes described herein for the preparation of crystalline Forms A or B of lacosamide uses lacosamide as starting material. Lacosamide used as starting material can be present in any polymorphic form. Crude lacosamide can be used as starting material. Preferably, lacosamide as starting material can be obtained by following the process described in the U.S. '551 patent. More preferably, the crystalline Form B of lacosamide can be used as starting material for the preparation of said Form A and the crystalline Form A of lacosamide can be used as starting material for the preparation of said Form B.

### BRIEF DESCRIPTION OF THE DRAWINGS

FIG. 1: X-ray powder diffraction pattern (XRPD) of Form A of lacosamide which is obtained by following the process described in the WO 2006/037574 application.

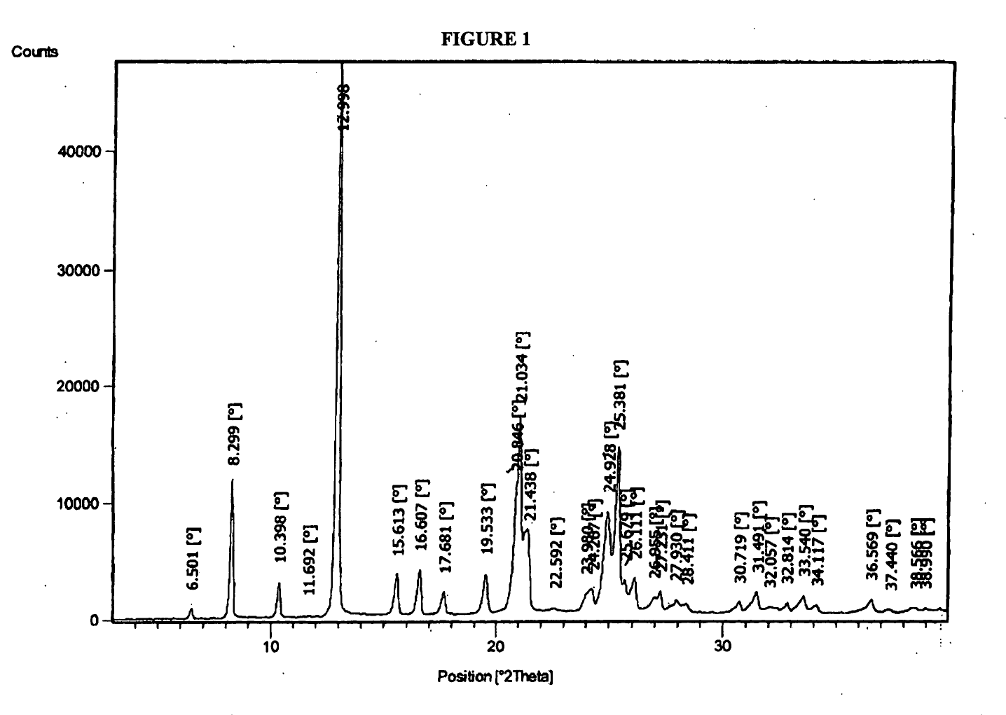

FIG. 2: X-ray powder diffraction pattern (XRPD) of Form A of lacosamide.

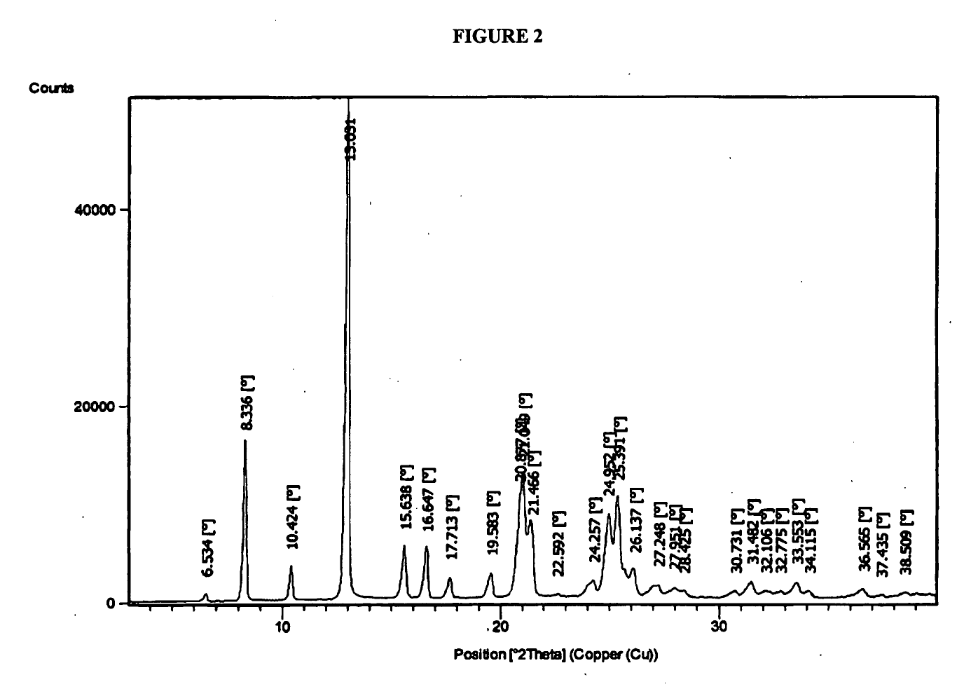

FIG. 3: Differential scanning calorimetric (DSC) thermogram of Form A of lacosamide.

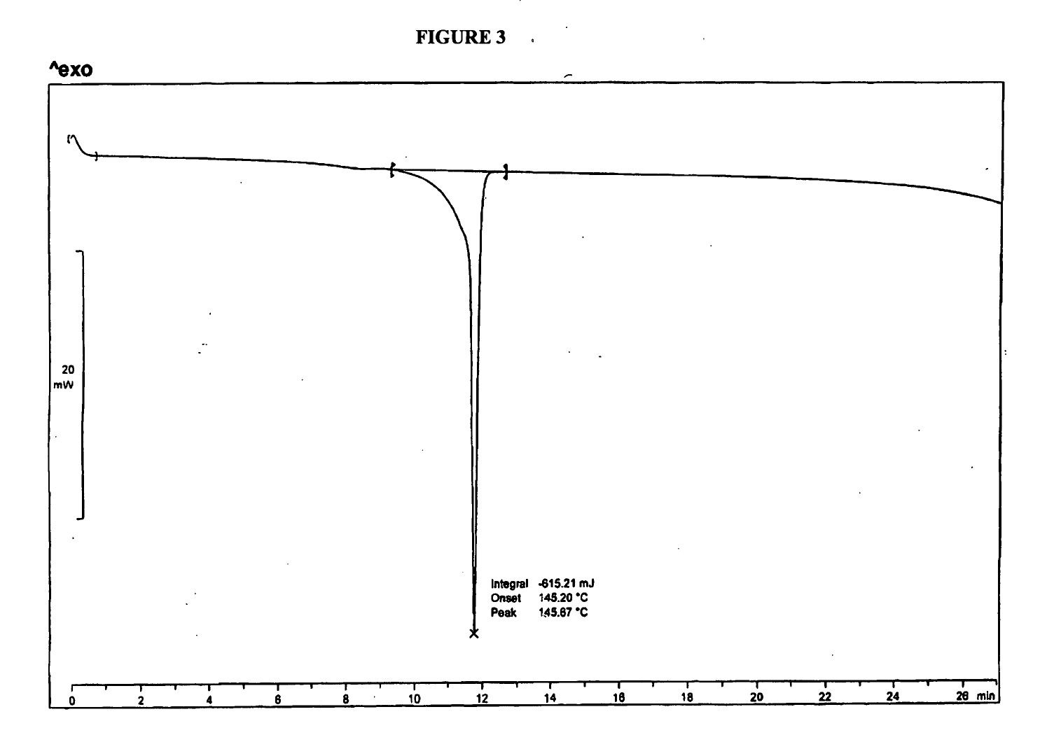

FIG. 4: X-ray powder diffraction pattern (XRPD) of Form B of lacosamide.

FIG. 5: Differential scanning calorimetric (DSC) thermogram of Form B of lacosamide.

FIG. 6: Infra-red spectrum (IR) of Form B of lacosamide.

The X-ray powder diffractograms (XRPD) were recorded on a PANalytical X'pert Pro instrument. The radiations at a measurement were done using CuK 45 kV.

Differential scanning calorimetric (DSC) thermograms was recorded on Mettler Toledo DSC 821 and Perkin Elmer Diamond DSC instrument.

### DETAILED DESCRIPTION OF THE INVENTION

The present invention can be explained by way of following aspects.

A first aspect of the present invention provides a process for the preparation of crystalline polymorphic Form A of lacosamide comprising crystallizing lacosamide with low volume of ethyl acetate.

In an embodiment of this aspect, the crystallization is performed at a temperature range of about −20° C. to about 35° C. Preferably, the crystallization is performed at a temperature range of about 0° C. to about 30° C.

Accordingly, lacosamide is admixed with a low volume of ethyl acetate, preferably, in an amount of about 7 to about 12 times by volume of ethyl acetate per gram of lacosamide, optionally through heating. If heating is performed, then depending on the amount of lacosamide and ethyl acetate used and the heating temperature may vary. Preferably, the heating can be performed at a temperature of about 50° C. to about 80° C. More preferably, the heating can be done at reflux temperature of the ethyl acetate. Solution or suspension may form even after heating depending upon the amount of ethyl acetate taken. The obtained solution or suspension is then cooled to between about 35° C. and about −20° C. to crystallize Form A of lacosamide followed by recovery of the said Form A through filtration, optionally followed by washing with ethyl acetate and/or drying.

A second aspect of the present invention provides a process for the preparation of crystalline polymorphic Form A of lacosamide comprising suspending lacosamide in water and isolating the Form A of lacosamide.

In an embodiment of this aspect, lacosamide is suspended in water through stirring at about 25° C. to about 40° C.

In another embodiment of this aspect, Form A of lacosamide is isolated from the said suspension through filtration followed by optional washing with water and/or drying.

Accordingly, lacosamide is suspended in de-ionized water through stirring at about 25° C. to about 40° C. The suspension is then filtered and the crystals obtained are washed with de-ionized water. The washed crystals are then dried to obtain said Form A of lacosamide.

A third aspect of the present invention provides a process for the preparation of crystalline polymorphic Form A of lacosamide comprising:

a) suspending lacosamide in organic solvent and/or water; and

b) recovering Form A of lacosamide from the suspension of step a).

In an embodiment of this aspect, the organic solvent is selected from the group comprising of ethyl acetate, acetone and methanol.

In another embodiment of this aspect, recovery of the Form A is performed at a temperature range of about −20° C. to about 35° C. Preferably, the recovery of Form A is performed at a temperature range of about 0° C. to about 30° C.

Accordingly, lacosamide is suspended in organic solvent and/or water optionally followed by stirring. The obtained suspension is then cooled to between about 35° C. and about −20° C. to obtain Form A of lacosamide followed by recovery of the said Form A through filtration, optionally followed by washing with organic solvent and/or drying.

In another embodiment of this aspect, the organic solvent is ethyl acetate.

In another embodiment of this aspect, the organic solvent is acetone.

In another embodiment of this aspect, the organic solvent is methanol.

In another embodiment of this aspect, lacosamide is suspended in water.

A fourth aspect of the present invention provides a process for the preparation of crystalline polymorphic Form A of lacosamide comprising crystallizing lacosamide with acetone.

In an embodiment of this aspect, the crystallization is performed at a temperature range of about −10° C. to about 10° C. Preferably, the crystallization is performed at a temperature range of about −5° C. to about 5° C.

Accordingly, lacosamide is dissolved in acetone, optionally through heating. The solution is then cooled to between about −10° C. and about 10° C. to initiate crystallization. The crystals obtained are filtered followed by optional washing with acetone and/or drying.

A fifth aspect of the present invention provides a process for the preparation of crystalline polymorphic Form A of lacosamide comprising crystallizing lacosamide with methanol.

In an embodiment of this aspect, the crystallization is performed at a temperature range of about −10° C. to about 20° C. Preferably, the crystallization is performed at a temperature range of about −5° C. to about 15° C.

Accordingly, lacosamide is dissolved in methanol optionally through heating. The solution is then cooled to between about −10° C. and about 20° C. to initiate crystallization. The crystals obtained are filtered, followed by optional washing with methanol and/or drying.

A sixth aspect of the present invention provides a process for the preparation of crystalline polymorphic Form A of lacosamide comprising crystallizing lacosamide with a mixture of organic solvents.

In an embodiment of this aspect, the mixture of organic solvent comprises ethyl acetate as one of the solvents and the amount of ethyl acetate taken is not more than about 12 times by volume per gram of lacosamide.

In another embodiment of this aspect, the mixture of organic solvent is ethyl acetate and toluene.

In another embodiment of this aspect, the mixture of organic solvent is ethyl acetate and dichloromethane.

In another embodiment of this aspect, the crystallization is performed at a temperature range of about −20° C. to about 35° C.

Accordingly, lacosamide is admixed with a mixture of organic solvents optionally through heating and/or stirring. The obtained solution or suspension is then cooled to between about 30° C. and about −20° C. to obtain crystals of Form A of lacosamide. The crystals are recovered through filtration optionally followed by washing and/or drying.

A seventh aspect of the present invention provides a process for the preparation of crystalline polymorphic Form A of lacosamide comprising crystallizing lacosamide with a mixture of methanol and hexanes.

In an embodiment of this aspect, the crystallization is performed at a temperature range of about −20° C. to about 30° C.

Accordingly, lacosamide is added to methanol, optionally using heating. Hexanes are added to the obtained solution. The resultant solution is then cooled to between about 30° C. to about −20° C. to obtain crystals of Form A of lacosamide. The crystals are recovered through filtration optionally followed by washing and/or drying.

An eighth aspect of the present invention provides a process for the preparation of crystalline polymorphic Form B of lacosamide comprising crystallizing lacosamide with high volume of ethyl acetate.

In another embodiment of this aspect, the crystallization is performed at temperature range of about −20° C. to about 45° C. Preferably, the crystallization is performed at temperature range of about 0° C. to about 30° C.

Accordingly, lacosamide is admixed with ethyl acetate, preferably, in an amount of 12 times or more by volume of ethyl acetate per gram of lacosamide, optionally through heating. If heating is performed, then depending on the amount of lacosamide and ethyl acetate taken, the heating temperature may vary. Preferably, the heating can be performed at a temperature of about 50° C. to about 80° C. More preferably, the heating can be done at reflux temperature of ethyl acetate. The obtained solution is then cooled to between about 45° C. and about −20° C. to crystallize the Form B of lacosamide followed by recovery of the said Form B through filtration, optionally followed by washing with ethyl acetate and/or drying.

A ninth aspect of the present invention provides a process for the preparation of crystalline polymorphic Form B of lacosamide comprising crystallizing lacosamide with toluene.

In an embodiment of this aspect, the Form B of lacosamide is crystallized from suspension of lacosamide in toluene.

In another embodiment of this aspect, the Form B of lacosamide is crystallized from the solution of lacosamide in toluene.

Accordingly, lacosamide is admixed with toluene optionally through heating. If suspension is formed, it is stirred at ambient temperature and then filtered to isolate crystals of Form B of lacosamide. If heating is applied to obtain solution of lacosamide in toluene, then the obtained solution is cooled to form crystals of Form B of lacosamide. The crystals are recovered through filtration and dried.

A tenth aspect of the present invention provides a process for the preparation of crystalline polymorphic Form B of lacosamide comprising crystallizing lacosamide with mixture of organic solvents.

In an embodiment of this aspect, the mixture of organic solvent comprises ethyl acetate as one of the solvents and the amount of ethyl acetate taken is 12 times or more by volume per gram of lacosamide.

In another embodiment of this aspect, the mixture of organic solvent is ethyl acetate and toluene.

In another embodiment of this aspect, the mixture of organic solvent is ethyl acetate and dichloromethane.

In another embodiment of this aspect, the mixture of organic solvent is ethyl acetate and hexanes.

In an embodiment of this aspect, the crystallization is performed at temperature range of about −20° C. to about 35° C.

Accordingly, lacosamide is admixed with mixture of organic solvents, optionally using heating. The obtained solution is then cooled to between about 30° C. and about −20° C. to obtain crystals of Form B of lacosamide. The crystals are recovered through filtration, optionally followed by washing and/or drying.

An eleventh aspect of the present invention provides a process for the preparation of crystalline polymorphic Form B of lacosamide comprising crystallizing lacosamide with a mixture of toluene and hexanes.

In an embodiment of this aspect, the crystallization is performed at a temperature range of about −20° C. to about 30° C.

Accordingly, lacosamide is added to toluene optionally through heating and/or stirring. Hexanes are added to the obtained solution. The resultant solution is then cooled to about 30° C. to about −20° C. to obtain crystals of Form B of lacosamide. The crystals are recovered through filtration optionally followed by washing and/or drying.

The crystalline polymorphic Form B of lacosamide can be characterized by X-ray powder diffraction peak at about 16.2±0.2° 2θ.

The crystalline polymorphic Form B can be further characterized by X-ray powder diffraction peaks at about 5.2, 10.8, 11.1, 12.6, 15.6, 20.6, 21.3, 21.7, 22.6, 23.3, 23.9 and 25.9±0.2° 2θ.

The crystalline polymorphic Form B can be further characterized by X-ray powder diffraction peaks at about 6.7, 8.23, 10.4, 17.5, 24.4, 27.2 and 27.8±0.2° 2θ.

The crystalline polymorphic Form B can be further characterized by X-ray powder diffraction peak having d-value (d-spacing) at about 5.5±0.2 Å.

The crystalline polymorphic Form B can be further characterized by X-ray powder diffraction peaks having d-values (d-spacing) at about 16.9, 8.2, 8.0, 7.0, 5.7, 4.3, 4.2, 4.1, 3.93656, 3.8, 3.7 and 3.4±0.2 Å.

The crystalline polymorphic Form B can be further characterized by X-ray powder diffraction peaks having d-values (d-spacing) at about 13.1, 10.8, 8.5, 5.1, 3.6, 3.3 and 3.2±0.2 Å.

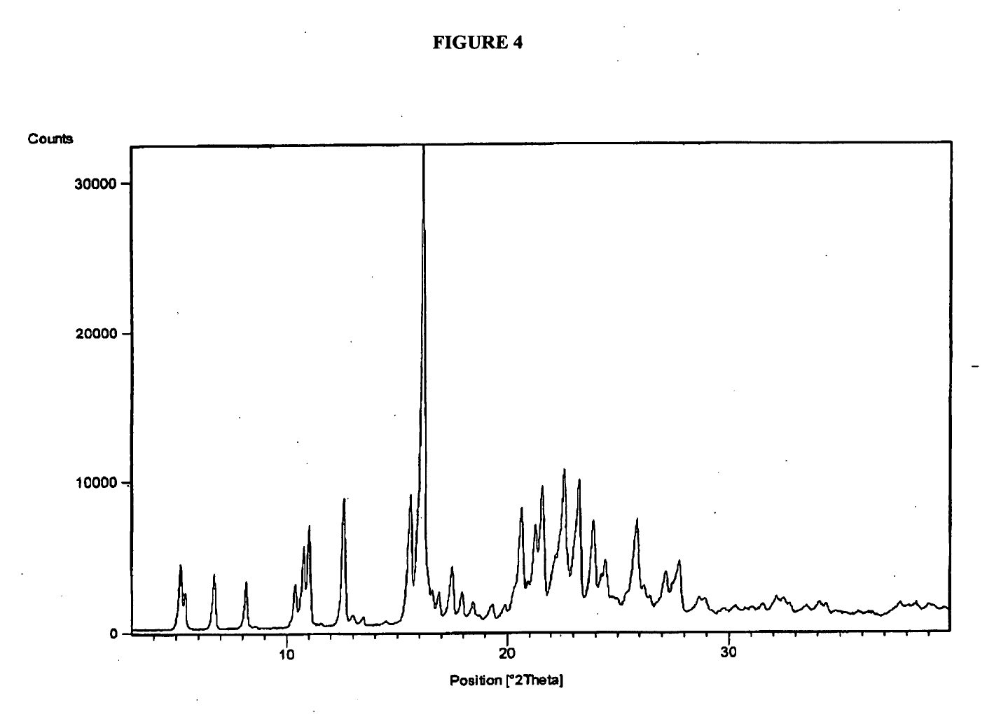

The crystalline polymorphic Form B of lacosamide can be characterized by X-ray powder diffraction (XRPD) pattern substantially as shown in FIG. 4.

The crystalline polymorphic Form B of lacosamide can be characterized by X-ray powder diffraction peaks expressed in 2θ angle, d-values (Å) and relative intensity (%) as given in Table 1.

TABLE 1
XRD peaks with d-spacings, relative intensity and 2θ angles for
Form B of lacosamide

| | | Rel. |
|---|---|---|
| Angle | d-values | Intensity |
| (2θ) | (Å) | (%) |
| 5.2 | 16.9 | 13.9 |
| 6.8 | 13.1 | 11.7 |
| 10.8 | 8.2 | 17.3 |
| 11.1 | 8.0 | 21.2 |
| 12.6 | 7.0 | 26.5 |
| 15.6 | 5.7 | 27.0 |
| 16.2 | 5.5 | 100.0 |
| 17.5 | 5.1 | 12.6 |
| 20.7 | 4.3 | 25.1 |
| 21.3 | 4.2 | 21.6 |
| 21.7 | 4.1 | 25.4 |
| 22.1 | 4.0 | 14.5 |
| 22.6 | 3.9 | 33.0 |
| 23.3 | 3.8 | 30.1 |
| 23.9 | 3.7 | 22.2 |
| 24.4 | 3.6 | 14.0 |
| 25.9 | 3.4 | 22.8 |
| 27.2 | 3.3 | 11.7 |
| 27.8 | 3.2 | 14.0 |

The crystalline Form B can be characterized by DSC thermogram comprising endothermic peak at about 145° C.

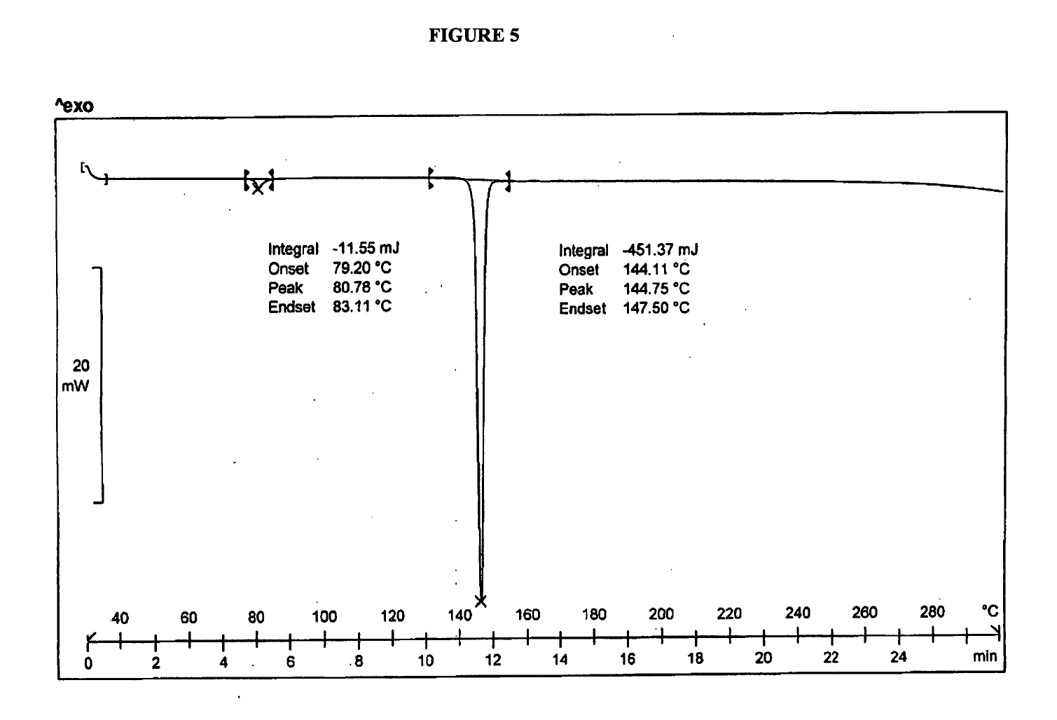

The crystalline Form B of lacosamide can be characterized by DSC thermogram substantially as shown in FIG. 5.

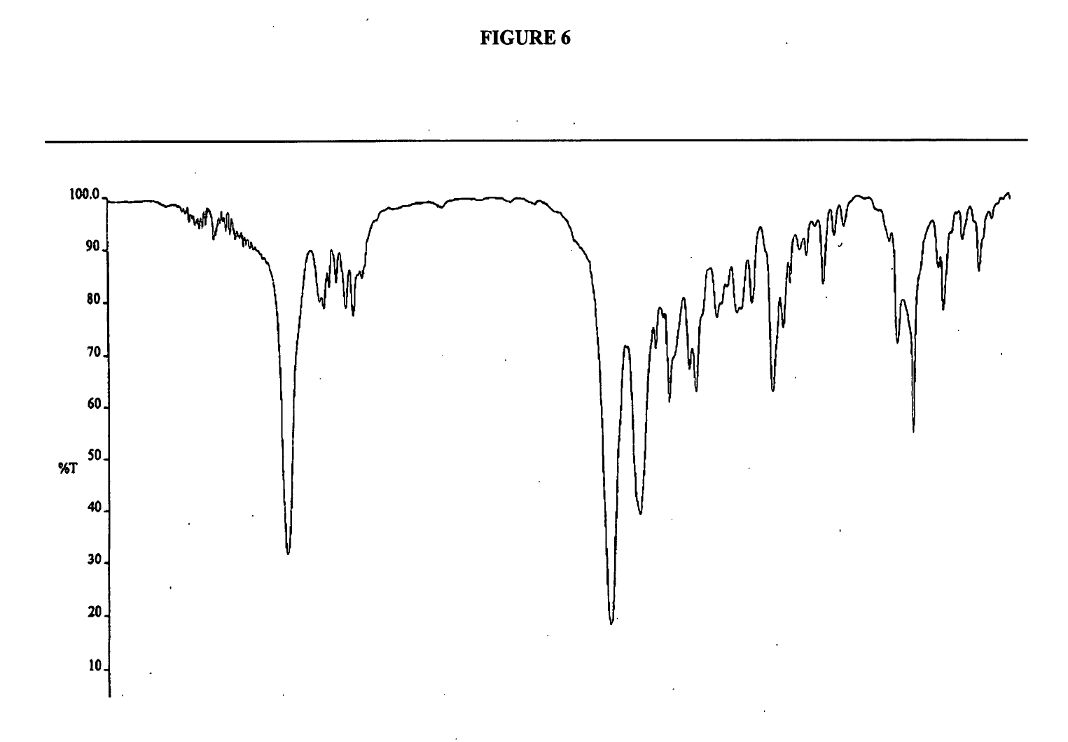

The crystalline Form B of lacosamide can be characterized by IR pattern substantially as shown in FIG. 6.

While the present invention has been described in terms of its specific aspects, certain modifications and equivalents will be apparent to those skilled in the art and are intended to be included within the scope of the present invention.

In the following section, aspects are described by way of examples to illustrate the processes of the invention and product thereof. However, the examples mentioned below are not intended in any way to limit the scope of the present invention. Several variants of these examples would be evident to persons ordinarily skilled in the art.

### Process For Preparation of Lacosamide

### Step 1: Preparation of N-trityl-D-serine

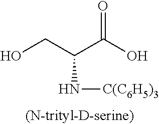

All operations were carried under inert atmosphere until reaction quenching.

To the suspension of D-serine (8.5 Kg) in dichloromethane (85 L) at ambient temperature, trimethyl silyl chloride (TMS.Cl; 10.5 Kg) was added in 15 to 30 minutes at 28° C. to 30° C. Reaction mass was stirred for 20 minutes under reflux. Hexamethyl disilazane (HMDS; 18.3 Kg) was added to it in 30 to 45 minutes under mild refluxing. It was further refluxed at 35° C. to 40° C. and stirred for 60 minutes under reflux at the same temperature. It was then cooled to 0° C. to −5° C. and at this temperature; solution of methanol diluted with dichloromethane (5.20 Kg in 17 L) was slowly added. The resultant solution was stirred for 15 minutes at 0° C. to 5° C. and then allowed it to reach at 20° C. to 30° C. Triethyl amine (8.16 Kg) was added to the solution and stirred for 10 minutes at 20° C. to 30° C. To this solution, trityl chloride (22.52 Kg) was added in various lots at 20° C. to 30° C. followed by aging for 3 hours at the same temperature. To the obtained reaction mixture, de-ionized water (26 L) was added and the layers formed were separated. The organic layer was washed with de-ionized water (26 L). Layers were separated and solvent was recovered from the organic layer to obtain crude material. To the crude material, toluene (17 L) was added at 50° C. and it was stirred. To the resultant solution, hexanes (43 L) were added at 40° C. to 45° C. It was stirred for 30 minutes at 35° C. to 40° C. The solution obtained was cooled to 0° C. to 5° C. and was stirred for 30 minutes. The solid formed was filtered and washed with hexanes (17 L). Drying under vacuum at 50° C. to 55° C. gave title compound 21.60 Kg.

### Step 2: Preparation of O-methyl-N-trityl-D-serine

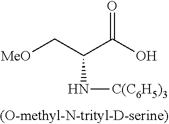

All operations were carried under inert atmosphere until reaction quenching.

To a suspension of imidazole (800 g) and sodium hydride (5 Kg) in tetrahydrofuran (100 L), trityl serine (step 1 product; 20 Kg) was added in lots at −15° C. to −10° C. The solution was stirred for 45 minutes at −15° C. to −10° C. and methyl iodide (16.4 Kg) was added. The temperature of the resultant reaction mixture was raised to −5° C. to 0° C. and then it was stirred for 3 hours at the same temperature. In other flask, de-ionized water (40 L) was kept at 0° C. to 5° C. The reaction mixture was transferred to the other flask containing de-ionized water at 0° C. to 5° C. Acetic acid (4.5 Kg) was added to the reaction mixture at 5° C. to 10° C. to neutralize aqueous layer (pH=6.5-7.0). Solvent was completely recovered under vacuum. The aqueous reaction mixture was cooled to ambient temperature and then dichloromethane (40 L) and de-ionized water (40 L) were added to the reaction mixture. The mixture was stirred, allowed to settle and the layers were separated. The organic layer was collected. The aqueous layer was extracted with dichloromethane (2×20 L), stirred and allowed to settle. The combined organic layers were washed with de-ionized water (40 L), dried over anhydrous sodium sulphate. The organic layer was filtered through a hyflo bed and the filtrate (dichloromethane layer) was collected.

Step 3: Preparation of N-benzyl-O-methyl-N²-trityl-D-serinamide

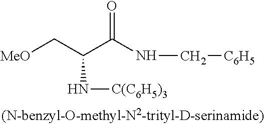

All operations were carried under inert atmosphere until reaction quenching.

The filtrate (dichloromethane layer) obtained as above was cooled to 15° C. to 20° C. and then N-methyl morpholine (NMM; 6.2 Kg) was added to it. The temperature of the resultant solution was allowed to reach at 20° C. to 25° C. The solution was then stirred for 15 minutes at this temperature (‘Solution-A’).

In another flask, isobutyl chloroformate (8.2 Kg) was added to dichloromethane (42 L) at ambient temperature. The solution was cooled to −10° C. to −15° C. and then stirred for 15 minutes at this temperature (‘Solution-B’).

‘Solution-A’ was added to ‘solution-B’ slowly at −15° C. to −10° C. and then stirred for 30 minutes at this temperature. A cooled solution of benzyl amine (7.40 Kg) in dichloromethane (20 L) was added to the mixture of ‘Solution-A’ and ‘Solution B’ slowly and stirred for 30 minutes at the same temperature. This reaction mixture was slowly heated to 20° C. to 25° C. and stirred for 2 hours at this temperature. The reaction mixture was further cooled to −10° C. to −15° C. and N-methyl morpholine (NMM, 1.6 Kg) was added to it in 10 to 15 minutes. It was stirred for 15 minutes at −10° C. to −15° C. and at this temperature, isobutyl chloroformate (IBCF; 2 Kg) was added to it. The reaction mixture was stirred further for 15 minutes at −10° C. to −15° C. and then a solution of benzyl amine (1.80 Kg) in dichloromethane (5 L) was added to the mixture at the same temperature. The mixture was allowed to reach at ambient temperature and then de-ionized water (42 L) was added to it. The mixture was stirred, allowed to settle and the layers separated. The organic layer was further washed with precooled solution of citric acid (2.4 Kg) in de-ionized water (42 L). The separated organic layer (dichloromethane layer) was collected.

### Step 4: Preparation of N-benzyl-O-methyl-D-serinamide

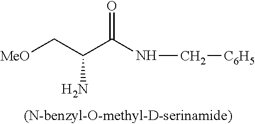

To the obtained organic layer as above, concentrated hydrochloric acid (6.2 Kg) was added at 25° C. to 30° C. in 15 to 20 minutes and the obtained reaction mixture was stirred for 60 minutes. De-ionized water (20 L) was added to the mixture and the layers were separated.

The organic layer was washed with de-ionized water (20 L). The aqueous layer was washed with dichloromethane (10 L) and hexanes (20 L), respectively. The obtained aqueous layer was cooled to 20° C. to 25° C. and then the pH of this aqueous layer was adjusted to 11 to 11.5 by adding solution of sodium hydroxide (2.60 Kg in 26 L de-ionized water) into it. The aqueous layer was washed with hexanes (20 L) and the product was extracted with dichloromethane (3×40 L). The organic layers were combined and washed with de-ionized water (40 L). The layers were separated and the organic layer (dichloromethane layer) was collected.

### Step 5: Preparation of Lacosamide

To the above organic layer, dimethyl amino pyridine (DMAP; 0.12 Kg) was added followed by addition of acetic anhydride (4.90 Kg) at 25° C. to 30° C. The reaction mass was stirred for 2 hours at the same temperature. The reaction mass was washed with de-ionized water (20 L) and the pH of the organic layer was adjusted to 6.5 to 7.5 using sodium hydroxide solution (2.31 Kg in 10 L de-ionized water) at 25° C. to 30° C. The organic layer was separated and activated carbon (0.49 Kg) was added to it. It was stirred for 15 minutes and then filtered through a hyflo bed. The bed was washed with dichloromethane (10 L) at 25° C. to 30° C. The solvent was recovered to obtain solid. Ethyl acetate (10 L) was added to the solid at 25° C. to 30° C. The solvent was again recovered at atmospheric pressure and at 40° C. The solid left was cooled to ambient temperature and ethyl acetate (84 L) was added to it. It was heated to reflux and the clear solution was obtained. It was further stirred for 5 minutes at reflux temperature. The solution obtained was passed through an 0.45 micron filter and the filter was washed with hot ethyl acetate (18 L). The solution obtained was heated to 70° C. to 75° C. and stirred for 5 minutes further at reflux temperature. The solution was then cooled to 30° C. in 1 hour. It was further cooled to 25° C. in 30 minutes. Toluene (30 L) was added to it and the resultant solution was stirred for 30 minutes at 25° C. to 30° C. The solution was cooled to 0° C. and stirred for 30 minutes at 0° C. to 5° C. The crystals formed were filtered and washed with toluene (20 L) at 5° C. to 10° C. The wet solid (crystals) were dried under vacuum at 60° C. to 65° C. The dried crystals were characterized as polymorphic Form B of lacosamide.

Dried weight: 5.80 Kg

### Yield: 38.68%

### Processes for Preparation of Polymorphic Forms of Lacosamide

### Example 1

Lacosamide (50 g) was added to ethyl acetate (300 ml) at ambient temperature and heated at 77° C. to 79° C. This was cooled to ambient temperature in 2 hours and the obtained suspension was filtered. The mass so obtained was dried at 60° C. to 65° C. to obtain crystals of Form A of lacosamide.

### Example 2

Lacosamide (50 g) was added to ethyl acetate (300 ml) at ambient temperature and heated at 77° C. to 79° C. This was cooled to ambient temperature in 2 hours followed by stirring for 1 hour at the same temperature. The suspension was further cooled to 0° C. to 5° C. in 1 hour followed by stirring for the next 1 hour at the same temperature. The suspension is then filtered and the crystals recovered were suck dried for 15 minutes. The crystals obtained were further dried under vacuum at 60° C. to 65° C. Dried crystals were characterized from its XRD as Form A of lacosamide.

### Yield: 90.52%

### Example 3

Lacosamide (20 g) was added to ethyl acetate (120 ml) at ambient temperature and heated at 75° C. to 80° C. This was cooled to ambient temperature in 1 hour and then filtered. The wet mass was characterized as Form A of lacosamide. The wet mass was dried at the following temperatures each for the specified time and analyzed.

The following were the results of drying:

| | | | Product characterized |
|---|---|---|---|
| | Temperature | Time | (based on XRD thereof) |
| SN | (° C.) | (in hours) | as |
| 1. | 35-40 | 6 | Crystalline Form A |
| 2. | 40-45 | 6 | Crystalline Form A |
| 3. | 45-50 | 6 | Crystalline Form A |
| 4. | 50-55 | 6 | Crystalline Form A |
| 5. | 55-60 | 6 | Crystalline Form A |
| 6. | 60-65 | 6 | Crystalline Form A |

Thus, there was no change in polymorph pattern even if it was heated for 36 hours in the temperature range of 35° C. to 65° C.

### Example 4

Lacosamide (15 g) was added to ethyl acetate (105 ml) at ambient temperature and heated at 75° C. to 80° C. This was cooled to 25° C. to 30° C. in 1 hour and then filtered. The crystals obtained were dried at 50° C. to 55° C. Dried crystals were characterized from its XRD as Form A of lacosamide.

### Example 5

Lacosamide (15 g) was added to ethyl acetate (105 ml) at ambient temperature and heated at 75° C. to 80° C. This was cooled to 25° C. to 30° C. in 1 hour and then further cooled to 0° C. to 5° C. The crystals formed were filtered and dried at 50° C. to 55° C. Dried crystals were characterized from its XRD as Form A of lacosamide.

### Yield: 79%

### Example 6

Lacosamide (5 g) was added to de-ionized water (25 ml) at 30° C. to 35° C. followed by stirring for 60 to 65 minutes at the same temperature. The suspension obtained was filtered and the crystals obtained were washed with de-ionized water (10 ml). The crystals were dried under vacuum at 60° C. to 65° C. Dried crystals were characterized from its XRD as Form A of lacosamide.

### Yield: 70%

### Example 7

Lacosamide (5 g) was added to de-ionized water (75 ml) at 30° C. to 35° C. followed by stirring for 55 to 60 minutes at the same temperature. The suspension obtained was filtered and the crystals obtained were washed with de-ionized water (10 ml). The crystals were dried under vacuum at 60° C. to 65° C. Dried crystals were characterized from its XRD as Form A of lacosamide.

### Yield: 34%

### Example 8

Lacosamide (10 g) was added to acetone (50 ml) at ambient temperature followed by stirring for 10 minutes. This was heated to reflux at 55° C. to obtain clear solution. The solution was cooled to ambient temperature in 1 hour followed by stirring for 30 minutes at 0.5 the same temperature. The solution was further cooled to 0° C. and stirred for the next 30 minutes at 0° C. to 5° C. The crystals so obtained was filtered and washed with chilled acetone (20 ml). The crystals were suck dried for 15 minutes followed by drying under vacuum or in air dryer at 50° C. to 55° C. Dried crystals were characterized from its XRD as Form A of lacosamide.

### Yield: 68%

### Example 9

Lacosamide (10 g) was added to methanol (25 ml) at ambient temperature and then heated to reflux until clear solution was achieved. The solution was then cooled to ambient temperature in 50 to 55 minutes. The solution was further cooled to 10° C. to 15° C. in 30 minutes. The product obtained was filtered and dried. Dried crystals were characterized from its XRD as Form A of lacosamide.

### Example 10

Lacosamide (10 g) was added to methanol (25 ml) at ambient temperature and then heated to reflux until clear solution was achieved. The solution was then cooled to ambient temperature in 50 to 55 minutes. The solution was further cooled to 10° C. to 15° C. in 30 minutes. Hexanes (50 ml) were added to the cooled solution in 5 minutes at 15° C. to 20° C. The obtained mixture was cooled to 0° C. to 5° C. followed by stirring for 1 hour at the same temperature. The product obtained was filtered and washed with hexanes (20 ml). The product was suck dried for 10 minutes and then dried under vacuum at 60° C. to 65° C. Dried crystals were characterized from its XRD as Form A of lacosamide.

### Yield: 52.3%

### Example 11

Lacosamide (60 g) was added to a mixture of ethyl acetate (420 ml) and dichloromethane (240 ml) followed by heating at 55° C. to 60° C. to obtain a clear solution. The solution was stirred for 10 minutes and then cooled to 30° C. in 1 hour. It was again stirred for 30 minutes at 25° C. to 30° C. and cooled to 5° C. in 30 minutes. The solution was further stirred at 0° C. to 5° C. for 30 minutes. The product obtained was filtered and washed with ethyl acetate (30 ml) followed by suck drying. The product was further dried at 60° C. to 65° C. Dried crystals were characterized from its XRD as Form A of lacosamide.

### Yield: 78%

### Example 12

Lacosamide (20 g) was added to dichloromethane (80 ml) at 15° C. to 20° C. and heated to 35° C. to 40° C. until clear solution was achieved. Ethyl acetate (120 ml) was slowly added to the solution at 35° C. to 40° C. The resultant solution was then cooled to ambient temperature in 45 minutes and stirred for 1 hour at the same temperature. The solution was further cooled to 0° C. to 5° C. in 1 hour and stirred at 0° C. to 5° C. for the next 1 hour. The product obtained was filtered, suck dried and further dried at 60° C. to 65° C. Dried crystals were characterized from its XRD as Form A of lacosamide.

### Yield: 75%

### Example 13

Lacosamide (150 g) was added to dichloromethane (600 ml) at ambient temperature and heated to 35° C. to 40° C. until clear solution was achieved. Ethyl acetate (1050 ml) was slowly added to the solution at 35° C. to 40° C. The resultant solution was then cooled to 25° C. to 30° C. in 60 minutes and then stirred for 1 hour. The solution was further cooled to 0° C. to 5° C. in 70 to 75 minutes. It was stirred at the same temperature for 1 hour. The crystals obtained were filtered, suck dried and further dried at 60° C. to 65° C. Dried crystals were characterized from its XRD as Form A of lacosamide.

### Yield: 78.13%

### Example 14

Lacosamide (5 g) was added to ethyl acetate (40 ml) at ambient temperature and heated to 55° C. to 60° C. The suspension obtained was stirred for 15 to 20 minutes at the same temperature and then cooled to ambient temperature in 55 to 60 minutes. The product obtained was filtered and dried. Dried crystals were characterized from its XRD as Form A of lacosamide.

### Example 15

Lacosamide (5 g) was added to ethyl acetate (40 ml) at ambient temperature and heated to 55° C. to 60° C. The suspension obtained was stirred for 15 to 20 minutes at the same temperature and then cooled to ambient temperature in 55 to 60 minutes. At this temperature, it was stirred for 25 to 30 minutes and further cooled to 0° C. to 5° C. in 25 to 30 minutes. The product obtained was filtered and dried. Dried crystals were characterized from its XRD as Form A of lacosamide.

### Example 16

Lacosamide (5 g) was added to ethyl acetate (40 ml) at ambient temperature and heated to 55° C. to 60° C. The suspension obtained was stirred for 15 to 20 minutes at the same temperature and then cooled to ambient temperature in 55 to 60 minutes. At this temperature, it was stirred for 25 to 30 minutes and further cooled to 0° C. to 5° C. in 25 to 30 minutes. Toluene (15 ml) was added to the suspension at 0° C. to 5° C. and stirred for 30 minutes at the same temperature. The product obtained was filtered and washed with toluene (10 ml) followed by suck drying. The product was further dried at 60° C. to 65° C. Dried crystals were characterized from its XRD as Form A of lacosamide.

### Yield: 89.2%

### Example 17

Lacosamide (10 g) was added to dichloromethane (70 ml) and heated to 28° C. to 30° C. The solution obtained was filtered through a hyflo bed followed by washing of the bed with dichloromethane (20 ml). The solvent was recovered under vacuum at 30° C. to 38° C. to obtain solid. The solid obtained was added to ethyl acetate (75 ml) at ambient temperature and heated to reflux. The solution so obtained was cooled to ambient temperature followed by stirring for 30 minutes at 20° C. to 25° C. Toluene (30 ml) was added to the solution at the same temperature. The resultant solution was then cooled to 5° C. followed by stirring for 30 minutes at the same temperature. The crystals so obtained were filtered, washed with chilled toluene (20 ml) and suck dried. The crystals were further dried at 60° C. to 65° C. Dried crystals were characterized from its XRD as Form A of lacosamide.

### Yield: 91%

### Example 18

Lacosamide (20 g) was added to methanol (40 ml) at ambient temperature and heated to 60° C. to 65° C. The solution was stirred at same temperature for 5 minutes and then hexanes (100 ml) were drop wise added to the solution in 5 to 10 minutes (temperature of the solution was dropped to 48° C. to 50° C.). The resultant solution was cooled to 25° C. to 30° C. and then to 0° C. to 5° C. At this temperature, the solution was stirred for 30 minutes. The crystals formed were filtered and washed with hexanes (40 ml). The crystals were suck dried for 30 minutes and further dried under vacuum at 60° C. to 65° C. Dried crystals were characterized from its XRD as Form A of lacosamide.

### Yield: 62%

### Example 19

Lacosamide (10 g) was added to ethyl acetate (145 ml) at ambient temperature and heated to 70° C. to 72° C. The solution was stirred for 15 minutes at same temperature and then cooled to 30° C. in 1 hour. The solution was further cooled to 0° C. to 5° C. in 30 minutes and stirred at the same temperature for 30 minutes. The crystals formed were filtered and washed with ethyl acetate (5 ml) at 0° C. to 5° C. The crystals were dried under vacuum at 60° C. to 65° C. and characterized from XRD thereof as Form B of lacosamide.

### Example 20

Lacosamide (50 g) was added to ethyl acetate (1 L) at ambient temperature and heated to 70° C. The hot solution was filtered and reheated to 70° C. The solution was then cooled to ambient temperature in 90 minutes. It was stirred for 30 minutes at the same temperature and further cooled to 0° C. to 5° C. To this solution, toluene (150 ml) was added at 0° C. to 5° C. and stirred foil hour at 0° C. to 5° C. The crystals formed were filtered and washed with toluene (25 ml) and dried. Crystals were characterized from XRD thereof as Form B of lacosamide.

### Yield: 64%

### Example 21

Lacosamide (10 g) was added to ethyl acetate (160 ml) at ambient temperature and heated to 70° C. The solution was stirred for 5 minutes at same temperature and then cooled to 30° C. in 1 hour. The solution was further cooled to 5° C. in 30 minutes and stirred at the same temperature for 30 minutes. The crystals formed were filtered and washed with ethyl acetate (5 ml) at 0° C. to 5° C. Crystals were characterized from XRD thereof as Form B of lacosamide.

### Yield: 79%

### Example 22

Lacosamide (10.5 kg) was added to ethyl acetate (166 L) at ambient temperature and heated to 70° C. The obtained solution was filtered under nitrogen through a micron filter and the micron filter was washed with ethyl acetate (10.5 L). The solution was heated to 70° C. and then cooled to 25° C. to 30° C. in 90 minutes. The solution was stirred for 30 minutes at the same temperature and then cooled to 0° C. to 5° C. in 60 minutes. Toluene (31.5 L) was added to the cooled solution and the solution was stirred for 30 minutes at 0° C. to 5° C. The crystals obtained were filtered and washed with chilled toluene (5.3 L). The crystals were dried under vacuum at 60° C. to 65° C. Dried crystals were characterized from its XRD as Form B of lacosamide.

### Yield: 80%

### Example 23

Lacosamide (10 g) was added to toluene (160 ml) at ambient temperature followed by stirring for 12 to 15 minutes. The resultant suspension was heated to 90° C. to 95° C. and then cooled to 60° C. to 55° C. The solution was stirred for 2 to 5 minutes at the same temperature and filtered to obtain crystals. Crystals were characterized from XRD thereof as Form B of lacosamide.

### Example 24

Lacosamide (10 g) was added to toluene (160 ml) at ambient temperature followed by stirring for 12 to 15 minutes. The resultant suspension was heated to 90° C. to 95° C. and then cooled to ambient temperature in 65 to 70 minutes and filtered to obtain crystals. Crystals were characterized from XRD thereof as Form B of lacosamide.

### Example 25

Lacosamide (25 g) was added to toluene (150 ml) at ambient temperature followed by stirring for 5 to 10 minutes. The resultant suspension was heated to 95° C. to 105° C. and then cooled to ambient temperature in 50 to 55 minutes. It was filtered to obtain crystals. Crystals were characterized from XRD thereof as Form B of lacosamide.

### Example 26

Lacosamide (10 g) was added to toluene (160 ml) at ambient temperature followed by stirring for 12 to 15 minutes. The resultant suspension was heated to 90° C. to 95° C. and then cooled to ambient temperature in 65 to 70 minutes. The solution was cooled further to 0° C. to 5° C. in 50 to 55 minutes. Hexanes (50 ml) were added to the solution in 5 to 10 minutes at 0° C. to 10° C. The resultant solution was stirred for 30 minutes at 0° C. to 5° C. The crystals so formed were filtered and washed with hexanes (40 ml). The crystals were dried under vacuum at 60° C. to 65° C. Dried crystals were characterized from its XRD as Form B of lacosamide.

### Example 27

Lacosamide (10 g) was added to ethyl acetate (180 ml) at ambient temperature and the resultant solution was heated to reflux temperature. The solution was then cooled to 60° C. to 55° C. At this temperature, hexanes (50 ml) were drop wise added to the solution. The solution was cooled to 25° C. to 30° C. and filtered to collect the crystals formed. The crystals were washed with hexanes (40 ml) and dried under vacuum at 60° C. to 65° C. Dried crystals were characterized from its XRD as Form B of lacosamide.

### Yield: 62%

### Example 28

Lacosamide (20 g) was added to dichloromethane (80 ml) at ambient temperature and heated to 38° C. to 42° C. until clear solution was achieved. Ethyl acetate (320 ml) was slowly added to the solution at 40° C. to 45° C. The resultant solution was then cooled to 25° C. to 30° C. in 60 to 65 minutes and stirred for 1 hour at the same temperature. The solution was further cooled to 0° C. to 5° C. in 1 hour and stirred at 0° C. to 5° C. for next 30 minutes. The product obtained was filtered, suck dried and further dried at 60° C. to 65° C. Dried crystals were characterized from its XRD as Form B of lacosamide.

### Example 29

Lacosamide (10 g) was added to ethyl acetate (145 ml) at ambient temperature and heated to 70° C. to 72° C. The solution was stirred for 15 minutes at same temperature and then cooled to 30° C. in 1 hour. The solution was further cooled to 0° C. to 5° C. in 30 minutes and stirred at the same temperature for 30 minutes. Toluene (30 ml) was added to the solution at 0° C. to 5° C. and stirred for 1 hour at the same temperature. The crystals obtained were filtered and dried under vacuum at 60° C. to 65° C. Dried crystals were characterized from its XRD as Form B of lacosamide.

### Example 30

Lacosamide (50 g) was added to ethyl acetate (1000 ml) at ambient temperature and heated to 70° C. The solution was then cooled to 30° C. in 1.5 hours. The solution was stirred for 30 minutes at ambient temperature and further cooled to 0° C. in 60 minutes. Toluene (150 ml) was added to the solution at 0° C. to 5° C. and stirred for 1 hour at the same temperature. The crystals obtained were filtered, washed with toluene (25 ml) and dried under vacuum at 60° C. to 65° C. Dried crystals were characterized from its XRD as Form B of lacosamide.

### Example 31

Lacosamide (10 g) was added to ethyl acetate (160 ml) at ambient temperature and heated to 70° C. The solution was stirred for 5 minutes at same temperature and then cooled to 30° C. in 1 hour. The solution was further cooled to 5° C. in 30 minutes. Toluene (30 ml) was added to the solution at 0° C. to 5° C. and stirred for 30 minutes at the same temperature. The crystals obtained were filtered, washed with toluene (10 ml) and dried under vacuum at 60° C. to 65° C. Dried crystals were characterized from its XRD as Form B of lacosamide.

### Yield=79%

### Example 32

Lacosamide (10 g) was added to ethyl acetate (200 ml) at ambient temperature and heated to 70° C. The solution was then cooled to 30° C. in 1 hour. The solution was further cooled to 0° C. to 5° C. Toluene (10 ml) was added to the solution at 0° C. to 5° C. and stirred for 30 minutes at the same temperature. The crystals obtained were filtered and dried under vacuum at 60° C. to 65° C. Dried crystals were characterized from its XRD as Form B of lacosamide.

### Yield: 76%

### Example 33

Lacosamide (10 g) was added to ethyl acetate (140 ml) at ambient temperature and heated to 70° C. The solution was then cooled to 30° C. in 1 hour. The solution was further cooled to 0° C. to 5° C. Toluene (10 ml) was added to the solution at 0° C. to 5° C. and stirred for 30 minutes at the same temperature. The crystals obtained were filtered and dried under vacuum at 60° C. to 65° C. Dried crystals were characterized from its XRD as Form B of lacosamide.

### Yield: 66%

### Example 34

Lacosamide Form B (20 g) was added to ethyl acetate (100 ml) at 30° C. to 35° C. and stirred for 1 hour at the same temperature. It was filtered to obtain crystals. The crystals were dried under vacuum at 60° C. to 65° C. and characterized from their XRD as Form A of lacosamide.

### Example 35

Lacosamide Form B (20 g) was added to ethyl acetate (100 ml) at 30° C. to 35° C. and stirred for 1 hour at the same temperature. At this stage, polymorph nature of produced crystals was checked by filtering a portion of the suspension and drying of the obtained crystals under vacuum at 60° C. to 65° C. (Form A of lacosamide). To the suspension, ethyl acetate (20 ml) was added at ambient temperature and stirred for 1 hour at the same temperature. Again, at this stage, polymorph nature of produced crystals was checked (found Form A of lacosamide). The process was continued with addition of ethyl acetate in lots of 20 ml until the volume of ethyl acetate became 10 times of batch size and the polymorph was checked after each addition (found Form A of lacosamide each time). Finally, the reaction mass was cooled to −15 to −20° C. in 30 to 40 minutes and stirred at the same temperature for 15 minutes. This was filtered and crystals obtained were washed with ethyl acetate (20 ml), suck dried and further dried at 60 to 65° C. The crystals obtained were characterized as Form A of lacosamide.

### Example 36

Lacosamide Form B (50 g) was added to ethyl acetate (300 ml) at ambient temperature and heated at 77° C. to 79° C. to obtain a suspension. This was cooled to ambient temperature in 2 hours. The suspension was filtered to obtain crystals of Form A of lacosamide.

### Example 37

Lacosamide Form B (50 g) was added to ethyl acetate (300 ml) at ambient temperature and heated at 77° C. to 79° C. to obtain a suspension. This was cooled to ambient temperature in 2 hours followed by stirring for 1 hour at the same temperature. The suspension was further cooled to 0° C. to 5° C. in 1 hour followed by stirring for next 1 hour at the same temperature. The suspension was then filtered and the crystals obtained were suck dried for 15 minutes. The crystals were further dried under vacuum at 60° C. to 65° C. Dried crystals were characterized from its XRD as Form A of lacosamide.

### Yield: 90.52%

### Example 38

Lacosamide Form B (5 g) was added to de-ionized water (25 ml) at 30° C. to 35° C. followed by stirring for 60 to 65 minutes at the same temperature. The suspension obtained was filtered and the crystals obtained were washed with de-ionized water (10 ml). The crystals were dried under vacuum at 60° C. to 65° C. Dried crystals were characterized from its XRD as Form A of lacosamide.

### Yield: 70%

### Example 39

Lacosamide Form B (5 g) was added to ethyl acetate (40 ml) at ambient temperature and heated to 55° C. to 60° C. The suspension obtained was stirred for 15 to 20 minutes at the same temperature and then cooled to ambient temperature in 55 to 60 minutes. The product obtained was filtered and dried. Dried crystals were characterized from its XRD as Form A of lacosamide.

### Example 40

Lacosamide Form B (5 g) was added to ethyl acetate (40 ml) at ambient temperature and heated to 55° C. to 60° C. The suspension obtained was stirred for 15 to 20 minutes at the same temperature and then cooled to 25° C. to 30° C. in 55 to 60 minutes. At this temperature, it was stirred for 25 to 30 minutes and further cooled to 0° C. to 5° C. in 25 to 30 minutes. The product obtained was filtered and dried. Dried crystals were characterized from its XRD as Form A of lacosamide.

### Example 41

Lacosamide Form B (5 g) was added to ethyl acetate (40 ml) at ambient temperature and heated to 55° C. to 60° C. The suspension obtained was stirred for 15 to 20 minutes at the same temperature and then cooled to 25° C. to 30° C. in 55 to 60 minutes. At this temperature, it was stirred for 25 to 30 minutes and further cooled to 0° C. to 5° C. in 25 to 30 minutes. Toluene (15 ml) was added to the suspension at 0° C. to 5° C. and stirred for 30 minutes at the same temperature. The product obtained was filtered and washed with toluene (10 ml) followed by suck drying. The product was further dried at 60° C. to 65° C. Dried crystals were characterized from its XRD as Form A of lacosamide.

### Yield: 89.2%

### Example 42

Lacosamide Form B (10 g) was added to dichloromethane (70 ml) and heated to 28° C. to 30° C. The solution obtained was filtered through hyflo bed followed by washing of the bed with dichloromethane (20 ml). The solvent was recovered under vacuum at 30° C. to 38° C. to obtain solid. The solid obtained was added to ethyl acetate (75 ml) at ambient temperature and heated to reflux. The solution so obtained was cooled to ambient temperature followed by stirring for 30 minutes at 20° C. to 25° C. Toluene (30 ml) was added to the solution at the same temperature. The resultant solution was then cooled to 5° C. followed by stirring for 30 minutes at 0° C. to 5° C. The crystals so obtained were filtered, washed with chilled toluene (20 ml) and suck dried. The crystals were further dried at 60° C. to 65° C. Dried crystals were characterized from its XRD as Form A of lacosamide.

### Yield: 91%

### Example 43

Lacosamide Form B (10 g) was added to methanol (25 ml) at ambient temperature and then heated to reflux until clear solution was achieved. The solution was then cooled to ambient temperature in 50 to 55 minutes. The solution was further cooled to 10° C. to 15° C. in 30 minutes. The product obtained was filtered and dried. Dried crystals were characterized from its XRD as Form A of lacosamide.

### Example 44

Lacosamide Form B (10 g) was added to methanol (25 ml) at ambient temperature and then heated to reflux until clear solution was achieved. The solution was then cooled to ambient temperature in 50 to 55 minutes. The solution was further cooled to 10° C. to 15° C. in 30 minutes. Hexanes (50 ml) were added to the cooled solution in 5 minutes at 15° C. to 20° C. The obtained mixture was cooled to 0° C. to 5° C. followed by stirring for 1 hour at the same temperature. The product obtained was filtered and washed with hexanes (20 ml). The product was suck dried for 10 minutes and then dried under vacuum at 60° C. to 65° C. Dried crystals were characterized from its XRD as Form A of lacosamide.

### Yield: 52.3%

### Example 45

Lacosamide Form B (20 g) was added to methanol (40 ml) at ambient temperature and heated to 60° C. to 65° C. The solution was stirred at same temperature for 5 to 10 minutes and then hexanes (100 ml) were drop wise added to the solution in 5 to 10 minutes (temperature of the solution was dropped to 48° C. to 50° C.). The resultant solution was cooled to 25° C. to 30° C. and then to 0° C. to 5° C. At this temperature; the solution was stirred for 30 minutes. The crystals formed were filtered and washed with hexanes (40 ml). The crystals were suck dried for 30 minutes and further dried under vacuum at 60° C. to 65° C. Dried crystals were characterized from its XRD as Form A of lacosamide.

### Yield: 62%

### Example 46

Lacosamide Form A (10 g) was added to ethyl acetate (145 ml) at ambient temperature and heated to 70° C. to 72° C. The solution was stirred for 15 minutes at the same temperature and then cooled to 30° C. in 1 hour. The solution was further cooled to 0° C. to 5° C. in 30 minutes and stirred at the same temperature for 30 minutes. Toluene (30 ml) was added to the solution at 0° C. to 5° C. and stirred for 1 hour at the same temperature. The crystals obtained were filtered and dried under vacuum at 60° C. to 65° C. Dried crystals were characterized from its XRD as Form B of lacosamide.

### Yield: 82%

### Example 47

Lacosamide Form A (10.5 kg) was added to ethyl acetate (166 L) at ambient temperature and heated to 70° C. The obtained solution was filtered under nitrogen through micron filter and the micron filter was washed with ethyl acetate (10.5 L). The solution was heated to 70° C. and then cooled to 25° C. to 30° C. in 90 minutes. The solution was stirred for 30 minutes at the same temperature and then cooled to 0° C. to 5° C. in 60 minutes. Toluene (31.5 L) was added to the cooled solution and the solution was stirred for 30 minutes at 0° C. to 5° C. The crystals obtained were filtered and washed with chilled toluene (5.3 L). The crystals were dried under vacuum at 60° C. to 65° C. Dried crystals were characterized from its XRD as Form B of lacosamide.

### Yield: 80%

### Example 48

Lacosamide Form A (10 g) was added to toluene (160 ml) at ambient temperature followed by stirring for 12 to 15 minutes. The resultant suspension was heated to 90° C. to 95° C. It was stirred for 2 to 5 minutes at 90° C. to 95° C. and then cooled to 60° C. to 55° C. The solution was stirred for 2 to 5 minutes at the same temperature and filtered to obtain crystals. Crystals were characterized from XRD thereof as Form B of lacosamide.

### Example 49

Lacosamide Form A (10 g) was added to toluene (160 ml) at ambient temperature followed by stirring for 12 to 15 minutes. The resultant suspension was heated to 90° C. to 95° C. It was stirred for 2 to 5 minutes at 90° C. to 95° C. and then cooled to ambient temperature in 55 to 60 minutes. The solution was stirred for 2 to 5 minutes and filtered to obtain crystals. Crystals were characterized from XRD thereof as Form B of lacosamide.

### Example 50

Lacosamide Form A (10 g) was added to toluene (160 ml) at ambient temperature followed by stirring for 12 to 15 minutes. The resultant suspension was heated to 90° C. to 95° C. and then cooled to ambient temperature in 55 to 60 minutes. The solution was cooled further to 0° C. to 5° C. in 50 to 55 minutes. Hexanes (50 ml) were added to the solution in 5 to 10 minutes at 0° C. to 10° C. The resultant solution was stirred for 30 minutes at 0° C. to 5° C. The crystals so formed were filtered and washed with hexanes (40 ml). The crystals were dried under vacuum at 60° C. to 65° C. Dried crystals were characterized from its XRD as Form B of lacosamide.

### Yield: 92.7%

### Stability Study of Polymorphic Form A of Lacosamide

### 1. Affect of Drying Temperature on Stability of Form A

Lacosamide polymorphic form A (having HPLC purity of 99.8%) was dried under following temperature and time profile.

| | Temperature | Time |
|---|---|---|
| SN | (° C.) | (hours) |
| 1. | 65-70 | 6 |
| 2. | 70-75 | 6 |
| 3. | 75-80 | 6 |
| 4. | 80-85 | 6 |
| 5. | 85-90 | 6 |
| 6. | 90-95 | 6 |

The following were observed:
a) There was no change in polymorphic form when the Form A was dried for 36 hours in temperature range of 65° C. to 95° C.
b) There was no change in chiral purity of the Form A (chiral purity was 100%).
c) Lacosamide Form A of HPLC purity 99.91% was obtained.

### 2. Affect of Time on Stability of Form A

Polymorphic Form A of lacosamide was kept at 0° C. to 5° C. for 3 months. After 3 months, it was reanalyzed and characterized as Form A of lacosamide.

### Stability Study of Polymorphic Form B of Lacosamide

### 1. Effect of Drying Temperature on the Stability of Form B

Lacosamide polymorphic form B (having HPLC purity of 99.85% and chiral purity of 100%) was dried under following temperature and time profile.

| | Temperature | Time |
|---|---|---|
| SN | (° C.) | (hours) |
| 1. | 65-70 | 6 |
| 2. | 70-75 | 6 |
| 3. | 75-80 | 6 |
| 4. | 80-85 | 6 |
| 5. | 85-90 | 6 |
| 6. | 90-95 | 6 |
| 7. | 95-100 | 6 |

The following were observed:
a) There was no change in polymorphic form when the form B was dried for 42 hours in temperature range of 65° C. to 100° C.
b) There was no change in chiral purity of the Form A (chiral purity was 100%).
c) Lacosamide Form B of HPLC purity 99.87% was obtained.

### 2. Effect of Time on Stability of Form B

Polymorphic Form B of lacosamide was kept at 0° C. to 5° C. for 7 months. After 7 months, it was reanalyzed and characterized as Form B of lacosamide.

### Solubility of Polymorphic Form A and Form B of Lacosamide in De-Ionized Water

Lacosamide polymorphic Forms A and B were tested for solubility studies by dissolving the Form A or Form B in de-ionized water. Results were as shown below:

| | Polymorphic Form | Solubility status in de-ionized water |
|---|---|---|
| SN. | of Lacosamide | (1 in 45 parts) |
| 1. | Form A | Sparingly soluble |
| 2. | Form B | Sparingly soluble |

1-26. (canceled)

27. A process for the preparation of crystalline polymorphic Form A of lacosamide comprising crystallizing lacosamide using an organic solvent selected from the group consisting of ethyl acetate, methanol, acetone, toluene, hexanes and mixtures thereof.

28. A process of claim 1 where in the organic solvent is a mixture of organic solvents.

29. A process of claim 1 wherein ethyl acetate is the organic solvent used at about 7 to about 12 times by volume of ethyl acetate per gram of lacosamide.

30. The process of claim 3 wherein the crystallization is performed at temperature range of about −20° C. to about 35° C.

31. The process of claim 1 wherein a mixture of organic solvents comprising ethyl acetate in an amount which is not more than about 12 times by volume per gram of lacosamide is used.

32. A process of claim 1 wherein acetone is the organic solvent.

33. A process of claim 6 wherein the crystallization is performed at temperature range of about −10° C. to about 10° C.

34. A process of claim 1 wherein methanol is the organic solvent.

35. The process of claim 8 wherein the crystallization is performed at temperature range of about −10° C. to about 20° C.

36. The process of claim 2 wherein the crystallization is performed at temperature range of about −20° C. to about 35° C.

37. The process of claim 2 wherein the mixture of organic solvents is a mixture of ethyl acetate with toluene or dichloromethane.

38. The process of claim 2 wherein the mixture of organic solvents is a mixture of methanol and hexanes.

39. A process for the preparation of crystalline polymorphic Form A of lacosamide comprising suspending lacosamide in water and isolating the Form A of lacosamide.

40. A process of claim 3, wherein an organic solvent is also used for suspending lacosamide.

41. The process of claim 4 wherein the organic solvent is selected from the group comprising of ethyl acetate, acetone and methanol.

42. A process for preparation of crystalline polymorphic Form B of lacosamide comprising crystallizing lacosamide using an organic solvent selected from the group consisting of ethyl acetate, methanol, acetone, toluene, hexanes and mixtures thereof.

43. A process of claim 16, wherein the organic solvent is toluene.

44. A process of claim 16, wherein the organic solvent is mixture of organic solvents.

45. The process of claim 18 wherein the crystallization is performed at temperature range of about −20° C. to about 35° C.

46. The process of claim 18 wherein the mixture of organic solvents is a mixture of ethyl acetate with toluene, dichloromethane or hexanes.

47. The process of claim 18 wherein a mixture of organic solvents comprising ethyl acetate in an amount which is at least 12 times by volume per gram of lacosamide is used.

48. The process of claim 21 wherein the mixture of organic solvents is a mixture of toluene and hexanes.
